(19) United States
(12) Patent Application Publication
(10) Pub. No.: US 2023/0355146 A1
(43) Pub. Date: Nov. 9, 2023

(54) SYSTEM AND METHOD OF DENTAL IMPLANT GLUCOSE MONITOR

(71) Applicant: Joseph John Parsi, Shavano Park, TX (US)

(72) Inventor: Joseph John Parsi, Shavano Park, TX (US)

(21) Appl. No.: 18/312,843

(22) Filed: May 5, 2023

Related U.S. Application Data
(60) Provisional application No. 63/339,176, filed on May 6, 2022.

(51) Int. Cl. A61B 5/1459; H02N 2/18; A61B 5/00; A61B 5/145

(52) U.S. Cl. CPC A61B 5/1459; H02N 2/186; A61B 5/0095; A61B 5/0031; A61B 5/14532; A61B 2560/0214

(57) ABSTRACT

A system for monitoring blood content, comprising an integrated laser circuit mounted on a tooth, an integrated detector circuit mounted on the tooth at a different location from the integrated laser circuit and a power supply coupled to the integrated laser circuit and the integrated detector circuit.

### RELATED APPLICATIONS

The present application claims benefit of and priority to U.S. provisional patent application 63/339,176, filed May 6, 2022, which is hereby incorporated by reference for all purposes as if set forth herein in its entirety.

### TECHNICAL FIELD

The present disclosure relates generally to glucose monitors, and more specifically to a system and method that uses a dental implant glucose monitor.

### BACKGROUND OF THE INVENTION

Monitoring blood for the presence of molecules such as glucose is difficult, because access to the blood must be sterile and cannot be easily obtained at the epidermal surface.

### SUMMARY OF THE INVENTION

A system for monitoring blood content is provided that includes an integrated laser circuit mounted on a tooth and an integrated detector circuit mounted on the tooth at a different location from the integrated laser circuit. A power supply is coupled to the integrated laser circuit and the integrated detector circuit, and the integrated detector circuit detects an indication of a molecule in the blood that enters the tooth pulp that is generated by exposure to a laser beam generated by the laser circuit.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of an endosteal implant, in accordance with an example embodiment of the present disclosure;

FIG. 2 is a diagram of an implant system, in accordance with an example embodiment of the present disclosure;

FIG. 3 is a diagram of a circuit for generating laser signals in vivo, in accordance with an example embodiment of the present disclosure;

FIG. 4 is a diagram of a circuit for measuring laser signals in vivo, in accordance with an example embodiment of the present disclosure;

FIG. 5 is a diagram of a contact area of an implant and natural tooth, which can be configured to provide power and means of direct communication, in accordance with an example embodiment of the present disclosure;

FIG. 6 is a diagram of an algorithm for configuration of components for monitoring blood glucose levels in vivo, in accordance with an example embodiment of the present disclosure; and

FIG. 7 is a diagram of an algorithm for monitoring blood glucose levels in vivo, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

The present application claims benefit of and priority to U.S. provisional patent application 63/339,176, filed May 6, 2022, which is hereby incorporated by reference for all purposes as if set forth herein in its entirety.

Near-infrared spectroscopy (NIR) has been used to continuously measure glucose level in the body, such as at surface epithelial tissues. The present disclosure is directed to systems and methods for using human teeth as a site for NIR glucose measurements. Photoacoustic spectroscopy using an NIR laser mounted on a molar tooth is able to continuously measure the blood glucose level in the open pulp chamber of the molar tooth. The specificity of the detection capability of the glucose molecule has been tested against other artificial commercial sweeteners and it has been shown that the photoacoustic spectroscopy on certain high absorbing wavelengths is very specific and can be used to detect the glucose molecule. Electronic components can be incorporated into the crown of the tooth and integrated into the oral maxillofacial system, and different power supply systems are also presented.

The present disclosure provides a novel closed-loop system for continuous glucose monitoring (CGM). The present disclosure uses blood for analysis of the glucose level, unlike existing CGM devices such as enzymatic devices that contain a needle that penetrate into epithelial tissue and remain there for a specific time, and optical devices also known as non-invasive or partially invasive devices that measure glucose through skin layers, based on optic properties such as absorption, scattering and transmittance and transmission. The enzymatic devices use interstitial fluid that is a dialysis of blood (less of plasma) under the skin while the optical devices use the skin interstitial fluid, though target small blood vessels, in fingers, stomach, earlobes, lips and tongue. There are many inaccuracies with optical devices stemming from the fact that none target the blood itself, which introduces a lack of accuracy due to nonspecific detection of glucose molecule in a mix of other tissue components. Furthermore, since the site of evaluation is on the skin, the process is affected by skin thickness and ambient temperature. On the other hand, the enzymatic detection under the skin, although very sophisticated and advanced and mostly accepted and used by the public, must accommodate a 5-20 minutes lag time from the time glucose is consumed to the time that it appears in the interstitial fluid. This is a significant problem for parents with children with type 1 diabetes or even for adult with diabetes type 2 who undergo a hypoglycemic episode. Often these individuals become unconscious due to low glucose in circulation, because glucose is an essential energy source for brain function.

The CGM device of the present disclosure overcomes these disadvantages with the prior art by being provided within a tooth crown that targets the blood vessel of molar tooth pulp with larger chamber where blood, glucose and other components of connective tissues circulate. In particular, the crown of the tooth is used to hold an electronic circuit that generates laser light having a specific wavelength that is sensitive to glucose, as well as multiple transducers such as photodiodes and piezoelectric devices for signal reception and transmission. The present disclosure utilizes principles of photoacoustic spectroscopy (PA) that have been shown to have a higher sensitivity and specificity for specific molecules, such as glucose. The power for the CGM crown is provided by a hollow implant that can be osseointegrated into the jaw bone next to the CGM crown. The hollow implant can carry batteries to support the function of the CGM crown, and a stack of piezoelectric devices is embedded into the crown of the hollow implant that can be used to harvest transmitted energy from the force of the mastication. The crown of the implant also contains communication circuitry to transfer the collected data from the CGM crown to a smart device for analysis.

Enamel on two opposing sides of the selected tooth can be removed up to the dentin, and two integrated circuits can be used to generate the blood glucose measurements: 1) a diode transmitter circuit secured to one side of the tooth, and 2) a photodiode circuit secured to the other side. A dense resin can be applied to block ambient light, where suitable.

The anatomy of human tooth provides an opportunity to measure blood glucose level at a very close proximity to blood while still retaining the sterility of the pulp chamber and its blood components and not invading the blood directly. It is composed of 1) enamel, a layer of 3-5 mm (hardest bone), 2) dentin, bone 3-7 mm (soft bone and alive), and 3) a pulp chamber (soft tissue filled with blood). It is a common practice to replace enamel with a porcelain, resin or metal crown, such as when the enamel becomes infected with bacteria and exposes the inner soft dentin to infection. To remove the damaged enamel and place a crown on the tooth, the enamel and a part of dentin are shaved off and removed. This procedure removes the infected or defective enamel while providing enough bulk for porcelain crown integrity (often about 2-3 mm). The remaining dentin on the tooth is often about 1-4 mm from the pulp chamber depending on the extension of infected enamel and dentin. Removing enamel and some dentin for the purpose of crown placement can thus also be used to implement a photoacoustic spectroscopy system for CGM into the manufactured crown. More specifically, a monochromatic optic laser can be embedded into a modified porcelain crown on one side that contains the embedded chip, to generate pulsated light at a certain power. The light passes through a thin layer of dentin that holds the pulp of the tooth containing blood, and a photoacoustic generated acoustic wave in the pulp can be detected by a transducer such as a piezoelectric circuit (PZT). The received weak signals from the PZT circuit are amplified and wirelessly transmitted to a smart device capable of analyzing the signal.

In one example embodiment, an NIR laser diode circuit having a wavelength of 950 nm can be attached to one side of a tooth and a photodiode circuit can be attached to the other side of the tooth. The NIR light passes through the enamel and dentin of the tooth and is detected by the photodiode.

The present disclosure provides a pulse modulator circuit that is connected to the NIR laser to produce a set of pulses. There is a higher peak-to-peak value in the detected light waveform as the concentration of glucose increased, and the shape of the peak remained the same. Laser photoacoustic spectroscopy can be used to detect a minute amount of a specific molecule within a medium, with a high degree of sensitivity. Application of this technique successfully on the tooth also relies on the selection of the correct transducers for the detection of acoustic pressure created by PA.

In PA, a laser beam is irradiated on a medium. The laser beam generates thermal expansion, leading to the generation of an acoustic wave. The generation of photoacoustic energy can take several forms, one where gaseous materials are detected, which requires a cell and a medium in between, and a second or direct form that does not require a cell. The modulated light source for photoacoustic generation can be from a continuous wave (which is not suitable for biological component, because the beam energy generates enough heat to damage tissue), or in a pulse format with short (e.g. nanosecond) to long (e.g. microsecond) pulse widths. When a pulsed laser is used, the wavelength of the PA wave that is generated is often less than one millimeter, which is within the biological component. The theory of photoacoustic data processing can be explained by volume thermal expansion and the equation of the motion:

$$∇ u = \frac{p}{ρ {v}^{2}} + βθ \qquad Eq . 1$$

$$\frac{ρ {d}^{2} u}{{dt}^{2}} = - ∇ p \qquad Eq . 2$$

Where:
u is the acoustic displacement vector
p is the acoustic pressure generated by thermoelastic expansion
ρ is the density of the medium
ν is the velocity of sound wave
β is thermal (cubic) expansion coefficient
θ is the temperature changes due to irradiation of optic beam

For the purpose of photoacoustic where the viscous effect and the thermal diffusion can be neglected, we can derive the following equation when combining the two-above equation:

$$( {∇}^{2} - \frac{1}{{v}^{2}} \frac{{d}^{2}}{{dt}^{2}} ) p = - \frac{β}{{C}_{p}} \frac{dh}{dt} \qquad Eq . 3$$

Where:
Cp is the specific heat of the medium
H is the function of the heat energy radiated on the medium

Knowing H, the energy that causes a change in the temperature of the medium and the fact that temperature depends on many other factors, it is not possible to derive to a general laser pulse shape for all the mediums. To derive a specific pulse shape, a cylindrical photoacoustic sources is assumed.

Under this scenario, the pulsed beam penetrates a long distance in the weakly absorbing medium, and eq. 3 can be modified to fit this condition:

$$( {∇}^{2} - \frac{1}{{v}^{2}} \frac{{d}^{2}}{{dt}^{2}} ) p = - \frac{α β}{{C}_{p}} \frac{dI}{dt} \qquad Eq . 4$$

Where:
I is the intensity of the laser pulse resulting in a temperature increase.

The current is of Gaussian form, hence:

$$P = \frac{α β \sqrt{v}}{Cp} \frac{k}{\sqrt{r} {Te}_{2}^{3}} F ( {t}^{′} / Te ) \qquad Eq . 5$$

Where:
α is the absorption coefficient of light
β is the thermoelastic expansion
ν is the velocity of sound in the medium
K is the amplitude
r is the detected distance of light penetration
F is a function of the shape of PA generation
t′ is the retarded time t′=(t−r/v)/Te
Where Te=\sqrt{Tp²+Ta²}, Tp is pulse width, and Ta is acoustic transit time.

From these equations, it can be deduced that if glucose changes the absorption of the light, it will result a change in α the optical absorption coefficient and this in turn changes the other physical parameters of the medium β, v and Cp. Therefore, the amplitude changes of PA pressure are defined by:

$$\frac{Δ P}{P} = \frac{Δ α}{α} ( 1 + \frac{Δ \frac{αβ \sqrt{v}}{Cp}}{\frac{β \sqrt{v}}{Cp}} ) + \frac{Δ \frac{αβ \sqrt{v}}{Cp}}{\frac{β \sqrt{v}}{Cp}} \qquad Eq . 6$$

This relationship is applicable for a laser pulse with a pulse width that is longer than the acoustic transit time. For a short pulse where the Tp>>Ta, the equation becomes:

$$\frac{Δ P}{P} = \frac{Δ α}{α} ( 1 + \frac{Δ \frac{αβ \sqrt{{v}^{2}}}{Cp}}{\frac{β \sqrt{{v}^{2}}}{Cp}} ) + \frac{Δ \frac{αβ \sqrt{{v}^{2}}}{Cp}}{\frac{β \sqrt{{v}^{2}}}{Cp}} \qquad Eq . 7$$

The relevant information that can be deduced from this equation is that the PA pressure wave is magnified as the absorption is increased as in the case of an increase in glucose concentration level. As glucose concentration reaches to its maximum biological level, the second term on the right-hand side of the equation increases by 0.01 or 1%, which reveals that PA is a better choice to determine the glucose level in the body than the optical absorption alone.

A planar photoacoustic source forms when the light beam radiates a highly absorbing or highly scattering medium to the point that the beam penetrates a small distance within the medium much smaller than the diameter of the beam itself. When a pulsed laser beam of short pulse width of intensity (I) irradiates over the medium of high absorbance; it follows the Beer's Law showing a degenerative curve of optical absorption. For this scenario, an applicable solution is:

$$P ( T ) = \frac{{E}_{0} α β {v}^{2}}{2 {C}_{p}} [ Θ ( - T 0 {e}^{α vT} + {R}_{c} Θ ( T ) {e}^{- α vT} ] \qquad Eq . 8$$

$${P}_{t} ( {T}_{t} ) = \frac{{E}_{0} α β {v}^{2}}{2 {C}_{p}} X Θ ( {T}_{t} ) {e}^{- α {v}_{t} {T}_{t}} \qquad Eq . 9$$

$$where$$

$${R}_{c} = ( {ρ}_{t} {v}_{t} - ρ v ) / ( {ρ}_{t} {v}_{t} + ρ v ) \qquad Eq . 10$$

$$X = ( 2 p {v}_{t} ) / ( {ρ}_{t} {v}_{t} - ρ v ) \qquad Eq . 11$$

$$T = t - \frac{z}{v} \qquad Eq . 12$$

$${T}_{t} = t - \frac{z}{{v}_{t}} \qquad Eq . 13$$

E₀ is the energy per unit area of laser beam
Θ(T) is the Heaviside unit function
ρ is the density
v is the acoustic speed in the highly absorbing medium
t subscript is the transparent medium

Eq. 9 identifies that all the waveforms are exponential with all heat function or optical absorbing medium. The right-side formula identifies that the first term is a forward going wave as the medium absorbs the light. The second term, however, is a reflection term. When there is a short pulse beam, such as (t<z/v), T<0, the second term is zero and only a forward wave can be detected. After time passes and (t>z/v), T>0, the reflective wave form presents itself in the second part of the wave shape, resulting in Rc (the reflection coefficient) in the highly absorbing medium.

This analysis can be used when the optically absorbing region in a medium is in a spherical shape, such as in droplets in a completely homogeneous transparent medium, or when the depth of penetration is equal to the radius of the beam wave. Under these scenarios, the shape of the sound wave is hemispherical. An equation for a Gaussian laser beam can be expressed as:

$$P ( r , t ) = \frac{E a β}{2 {π}^{3} {CpT}_{e}^{2} r} ( t - \frac{\frac{r}{v}}{Te} ) exp \{ - ( t - \frac{\frac{r}{v}}{Te} ) \} \qquad Eq . 14$$

From this equation, it can be seen that the acoustic pressure with peaks in positive and negative region for a short pulse can be defined as:

$$P ( r ) = K \frac{α β {v}^{2}}{Cp} exp ( - ( \frac{{r}^{2}}{Ra} ) ) \qquad Eq . 15$$

And for a longer pulse as:

$$P ( r , t ) = \frac{{K}^{′}}{r} \frac{α β}{Cp} ( t - \frac{\frac{r}{v}}{Tp} ) exp \{ - {( t - \frac{\frac{r}{v}}{Tp} )}^{2} \} \qquad Eq . 16$$

Where:
Tp is the pulse width for the laser pulse
K′ is a constant that includes the energy of laser pulse
For a short laser pulse, the change in acoustic pressure due to change in absorption can be written as:

$$\frac{Δ P}{P} = \frac{Δ E α}{E α} ( 1 + \frac{Δ \frac{β {v}^{2}}{Cp}}{\frac{β {v}^{2}}{Cp}} - 3 Δ Ra / Ra ) + \frac{Δ \frac{β {v}^{2}}{Cp}}{\frac{β {v}^{2}}{Cp}} - 3 Δ Ra / Ra \qquad Eq . 17$$

The maximum sensitivity can be determined when the ΔE and Δβν²/Cp are positive while ΔRa is negative. Glucose also intensifies the divergent effect between the reflective indices of cuvette and RBC in plasma resulting an increase in the Gruneisen Parameter, giving ΔE and Δβν²/Cp a positive value. This finding explains the fact that the PA acoustic pressure for glucose shows a higher sensitivity in whole blood then in the water.

PZT can be formed using a crystal lattice that becomes electrically polarized when subject to mechanical pressure. The deformation due to an exerted force will cause a dimensional change on the scale of nanometers, leading to a change in voltage on the scale of microvolts. Even though the voltage change is minute, utilizing an amplifier allows detection of small vibrational forces, thus making a PZT a good choice in many biological sensing devices. The successful performance of PZT depends at least in part on the electric and acoustic impedance matching.

Piezoelectric ceramic transducers can be used to detect a direct PA signal, mostly in solids and liquids. Some of the examples of this type of material are lead zirconate titanate PZT, lithium niobate, lead metaniobate, and crystal quartz. These transducers are useful for condensed matter because of good acoustic impedance matching (the acoustic transmission exceeds 50% for solids and 10% for liquids). PZT-5A or the soft version is suitable for biomedical devices.

A second kind of transducer is the polymeric films such as polyvinylidene difluoride (PVF2), which has a fast rise time, good flexibility and good acoustic impedance matching, especially to water and liquid in general. Its sensitivity is much less than the conventional PZT or even lithium niobate.

Another transducers is the capacitance transducer, which is generally formed from two plates with a dielectric sandwiched in between. The probe (one plate) is disposed close enough to the surface of the solid to receive the pressure wave, which is transmitted to the plate to create a change in the voltage at the dielectric site.

Fiber optics can be used to detect pressure and temperature. In this case, the fiber is formed into a coil and photoacoustic pressure impinging upon the coil will cause a change in the refractive index in the fiber resulting in a phase change in the propagation of the light hence a change in the physical state.

It is known that bones exhibit piezoelectric properties, therefore, as stress is applied to their structure, they produce a current within themselves. Conversely, if a current is applied to bone, it will cause the bone to compress. Bones also are dielectric, implying that they exhibit semiconductor electrical properties. They are also anisotropic, meaning they conduct the flow of current in certain path. Dentin is a different kind of bone.

In PA, NIR electromagnetic wave energy will cause the vibration of the molecular level at the atomic bonding sites, such as in water O—H and in glucose O—H and C—H. This vibration generates a thermo-elastic process that upon non-radiating relaxation will generate an acoustic pressure onto the surrounding medium, leading to an acoustic sound wave energy that can be detected by placing a transducer in contact with the medium. The intensity of the wave is a reflection of the absorption of light by the molecules in the solution and its conversion into sound energy. Therefore, if all the variables are kept in constant, the amplitude of the signal generated from PZT transducer will be a reflection of the concentration of the molecule in the solution and in this case, the concentration of glucose.

Photoacoustic spectroscopy for determining the level of an analyte in blood follows the basic formula of:

$$P = k ( \frac{{E}_{0} α β v}{Cp} ) \qquad Eq . 7-1$$

Where k is a constant of the detection system, E₀ is the laser pulsed energy and α, β, ν optical absorbent coefficient, thermal expansion coefficient and velocity of the sound in a solution and Cₚ is the specific heat constant. In optical measurements, optical scattering components in a turbid medium such as blood potentially limits its resolution and accuracy. For pulsed photoacoustic energy, the light still scatters but the optical absorption continues. In other words, the overall energy remains constant. Therefore, it is the most promising phenomena for a non-invasive measurement of blood glucose level.

In addition, this formula reveals the fact that the generation, propagation and magnitude of the signal and its visual inspection in the form of the wave can be predicted. Furthermore, many variables of the medium such as optical absorption α, thermoelastic β, or velocity of acoustic wave ν and even specific heat Cρ, all will affect the shape and magnitude of the wave. Glucose concentration also has significant effect on the same parameters α, β, and ν and Cρ. It has been determined that an increase of 1% glucose concentration will cause an increase of 1.2% in thermal expansion coefficient β, 28% increase in acoustic velocity ν, and a decrease of −0.6% in heat capacity Cₚ of the medium. For whole blood, the optical absorption coefficient is reported to be 0.7/mm with reduced scattering of 1.2/mm at the wavelength of 905 nm. The source of photoacoustic generation is of a more spherical shape with a radius of light of 0.5 mm, where the dominant factor affecting the change in photoacoustic signal is the change in the radius of the acoustic source.

It is not difficult to understand the rationale behind the dominant factor, namely, the radius of the source. The major acoustic source for PA in blood is red blood cells (RBC) with the size of 7.5 um in diameter and 2.5 um thickness. In a hyperglycemic condition (shock) when the glucose is first added to the blood mixture, RBC absorbs the glucose due to the osmolarity difference within the cell and the extracellular environment. This absorption results in an increased size of the RBC by 10-15%.

The amount of RBC is not constant in all individuals. Hematocrit is the level of RBC in blood and is important, as it effects are more profound on the PA waveform. It has been reported that there is a direct correlation of increased hematocrit number and type 2 diabetes. This correlation also contributes to higher viscosity of the blood. All of these facts (increase in the size of RBC, increase in the number of RBC in diabetes and higher viscosity) have a direct effect on the optical absorbance coefficient, optical scattering coefficient, velocity of the sound wave and the generation of acoustic pressure source in PA.

When the piezoelectric transducer is bonded to the tooth, it is outside of the chamber of blood separated by about 1 mm bone but in touch with dentin and held by a composite on the back. Because dentin (bone) is itself is a natural transducer, it can provide a coupling engagement and may increase the transducer's potential to receive signals from all around the molar tooth.

The present disclosure provides a CGM that uses PA for the measurement of glucose concentration in the pulp chamber of a tooth. The pulp chamber is surrounded by dentin, which acts as a cell in the way that most PA are utilized. Dentin is a form of bone and well known that bone is a form of piezoelectric. Therefore, the dentin walls of the pulp chamber help to conduct and couple acoustic pressure to the piezoelectric transducer. PA is the most precise measuring system capable of determining trace amount of chemical (glucose) in a medium. The pulse width of PA has a significant effect on the amplitude of the waveform that is used to determine the concentration of glucose in medium. Other optical properties such as absorption can be an adjunct in determining glucose concentration.

A titanium implant can be placed into the jaw bone for the replacement of missing tooth. The jaw bone is a combination of cortical bone outside and cancellous bone inside with high level of blood supplies. The blood provides the necessary minerals and nutrition for the implant to osseointegrate with the bone. This process will lead to a stable implant that can receive the force of mastication often about 70-150 Newtons or 16-34 lbf. The osseointegration process requires only the outer surface of the implant. Therefore, the inner portion can be hollowed to receive batteries to supply energy for the smart tooth. The battery can be made up of commercial circular batteries rechargeable lithium batteries or other suitable devices.

Piezoelectric materials generate electrical current in response to an applied force. The electrical energy is greater when piezoelectric devices are layered, such as by being stacked on top of each other. Utilizing this physical property of piezoelectric devices, stacks of piezoelectric devices can be brought together into several thin layers and embedded into the surface of the crown. A tooth with stacked piezoelectric devices can be strategically placed adjacent to the implant or placed directly on the implant crown, to recharge batteries inside the implant. The force of mastication thus generates electrical energy that can be harvested to recharge the batteries in the implant.

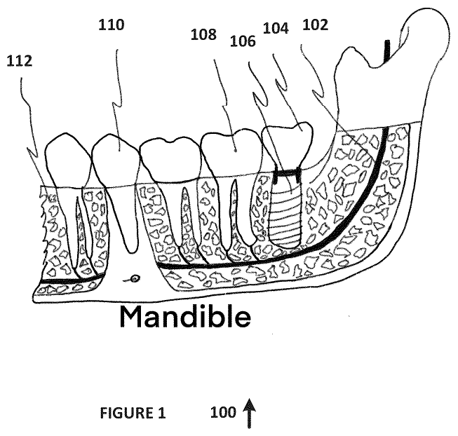

FIG. 1 is a diagram 100 of an endosteal implant 106, in accordance with an example embodiment of the present disclosure. Diagram 100 includes mandibular nerve and blood vessels 102, implant crown 104, endosteal implant 106, natural molar tooth 108, natural premolar tooth 110 and mandibular jaw bone 112. As discussed, endosteal implant 106 can be fabricated from titanium, epoxy, integrated circuits, piezoelectric devices and other components, and is implanted into mandibular jaw bone 112 adjacent to natural molar tooth 108, which can be modified to allow blood that is provided by mandibular nerve and blood vessels 102 to be tested for glucose content. Implant crown 104 can contain piezoelectric devices, a battery and other suitable components, as discussed and described in further detail herein.

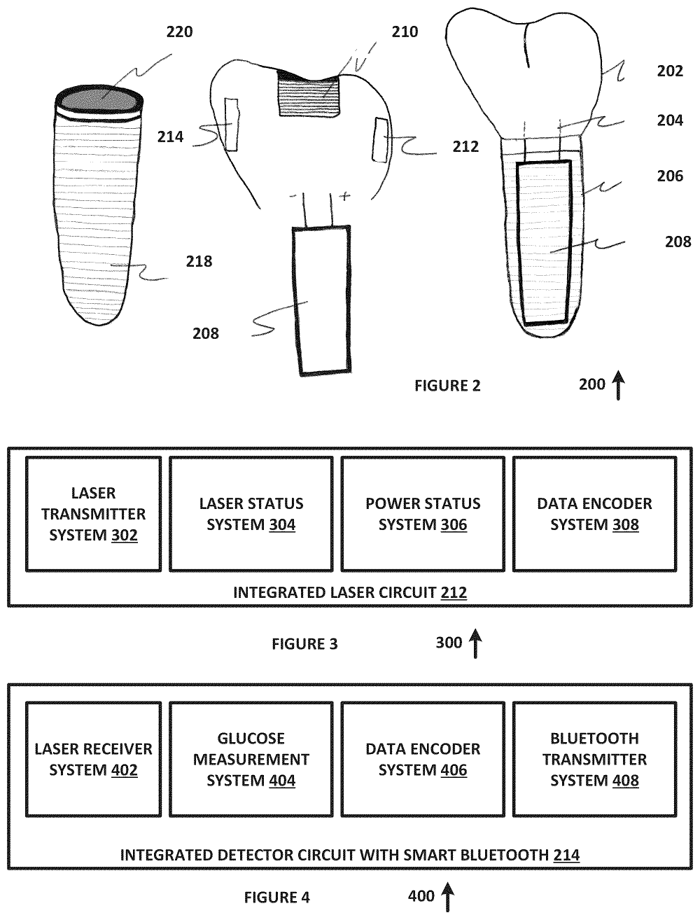

FIG. 2 is a diagram of an implant system 200, in accordance with an example embodiment of the present disclosure. Implant system 200 includes an implant crown 202, positive and negative leads 204 of battery or super capacitor 208, body of implant 206, integrated laser circuit 212, integrated detector circuit with smart Bluetooth 214, stacks of piezoelectric generators 210, body of implant 218, and hollow inside housing a battery or super capacitor 220.

Implant crown 202 can be formed from ceramic, epoxy or other suitable materials and can be configured to receive piezoelectric devices, such as stacks of piezoelectric generators 210 or other suitable devices. Implant crown 202 can be configured to be removable from body of implant 206, and can also include one or more leads to connect power stored in battery or super capacitor 220 to integrated laser circuit 212 and integrated detector circuit with smart Bluetooth 214.

Positive and negative leads 204 couple the piezoelectric devices of implant crown 202 to battery or super capacitor 220, to contacts of an adjacent live tooth that has integrated laser circuit 212 and integrated detector circuit with smart Bluetooth 214 incorporated into it, or to other suitable systems and components. Positive and negative leads 204 can be configured to detach from battery or super capacitor 220 to allow a practitioner to remove implant crown 202 and to service or replace battery or super capacitor 220 or other system components.

Battery or super capacitor 208 is configured to fit inside of the body of implant 206, and can store sufficient energy to power laser pulses of integrated laser circuit 212, to power integrated detector circuit with smart Bluetooth 214 to allow it to read and process data and to generate data packets for transmission, and to perform other suitable functions.

Integrated laser circuit 212 can be implemented in hardware or a suitable combination of hardware and software, and can be one or more integrated logic devices, data memory devices, NIR laser devices and other suitable devices. In one example embodiment, integrated laser circuit 212 can include additional circuitry to monitor power supply operating parameters, to detect when an incipient power supply failure is occurring, to detect when an laser device failure is occurring, to encode data messages to be transmitted with or instead of processed NIR data that indicates levels of glucose, and can perform other suitable functions. Integrated laser circuit 212 can be encapsulated to protect it from the harsh operating environment.

Integrated detector circuit with smart Bluetooth 214 can be implemented in hardware or a suitable combination of hardware and software, and can be one or more integrated PA detectors, logic devices, data memory devices, Bluetooth data communications devices and other suitable devices. In one example embodiment, integrated detector circuit with smart Bluetooth 214 can process detected NIR data using a PA detector to determine glucose levels, and can include additional circuitry to monitor power supply operating parameters, to detect when an incipient power supply failure is occurring, to detect when a Bluetooth communication device failure is occurring, to encode data messages to be transmitted with or instead of processed NIR data that indicates levels of glucose, and can perform other suitable functions. integrated detector circuit with smart Bluetooth 214 can be encapsulated to protect it from the harsh operating environment.

Stacks of piezoelectric generators 210 can be disposed on an implant, a live tooth or in other suitable locations to provide power for components of implant system 200. In one example embodiment, a single stack of piezoelectric generators 210 can be used in conjunction with an implant for power generation and storage, but stacks of piezoelectric generators 210 can also or alternatively be disposed on other tooth surfaces as needed, to receive and convert force to electrical energy as discussed and described in further detail herein. In addition to the uses disclosed herein, stacks of piezoelectric generators 210 can be used to provide energy for a triggering electrode to different part of brain for neurological diagnosis and treatment, to provide a source of power for the heart pacemakers, to provide a source of power for a drug delivery system or for other suitable purposes.

Body of implant 218 can be formed from titanium or other suitable materials, and can include a hollow inside housing a battery or super capacitor 220. Other suitable components can also or alternatively be disposed in body of implant 218, and body of implant 218 can be configured to maintain a sterile environment during operation.

In operation, implant system 200 allows blood glucose measurements to be made using NIR and PA devices in conjunction with a live tooth. Implant system 200 includes power generation, NIR laser, PA detector, Bluetooth communication and other suitable components in a flexible configuration, to allow a practitioner to configure implant system 200 in a manner that optimizes its operation without causing pain or discomfort to a patient.

FIG. 3 is a diagram of a circuit 300 for generating laser signals in vivo, in accordance with an example embodiment of the present disclosure. Circuit 300 includes integrated laser circuit 212 and NIR laser transmitter system 302, laser status system 304, power status system 306 and data encoder system 308, each of which can be implemented in hardware or a suitable combination of hardware and software.

NIR laser transmitter system 302 can be implemented using one or more integrated digital or analog devices in silicon, gallium arsenide, CMOS or other suitable materials, and can be configured to control laser power, pulse generation sequencing and data, frequency and other variables. In one example embodiment, NIR laser transmitter system 302 can be configured to operate whenever an amount of stored energy in an energy storage device reaches a level sufficient to power an NIR laser, and can include one or more oscillators, counters, data memory devices, encoders or other suitable components.

Laser status system 304 can be implemented using one or more integrated digital or analog devices in silicon, gallium arsenide, CMOS or other suitable materials, and can be configured to generate status data, such as when laser power consumption starts to increase and indicates potential failure mode, when NIR laser transmitter system 302 fails, and to provide other suitable functions. In one example embodiment, laser status system 304 can be configured to detect when a voltage across an impedance is lower than a threshold, which can indicate an incipient component failure, and can store a sequence of digital data values in a register that indicates the incipient component failure, where the digital data values can be encoded and transmitted by a Bluetooth communications system or in other suitable manners.

Power status system 306 can be implemented using one or more integrated digital or analog devices in silicon, gallium arsenide, CMOS or other suitable materials, and can be configured to generate status data, such as when voltage source current or voltage starts to decrease and indicates potential failure mode, when a piezoelectric power converter fails, and to provide other suitable functions. In one example embodiment, power status system 306 can be configured to detect when a voltage across an impedance is lower than a threshold, which can indicate an incipient component failure, and can store a sequence of digital data values in a register that indicates the incipient component failure, where the digital data values can be encoded and transmitted by a Bluetooth communications system or in other suitable manners.

Data encoder system 308 can be implemented using one or more integrated digital or analog devices in silicon, gallium arsenide, CMOS or other suitable materials, and can be configured to encodes data that indicates equipment status and warnings and to provide other suitable functions. In one example embodiment, data encoder system 308 can be configured to read digital data stored in memory buffers and to encode the data for transmission by a Bluetooth communications system or in other suitable manners.

FIG. 4 is a diagram of a circuit 400 for measuring laser signals in vivo, in accordance with an example embodiment of the present disclosure. Circuit 400 includes integrated detector circuit with smart Bluetooth 214 and laser receiver system 402, glucose monitor system 404, data encoder system 406 and Bluetooth transmitter system 408, each of which can be implemented in hardware or a suitable combination of hardware and software.

Laser receiver system 402 can be implemented using one or more integrated digital or analog devices in silicon, gallium arsenide, CMOS or other suitable materials, and can be configured to generate PA data, such discussed and described herein. In one example embodiment, laser receiver system 402 can be configured to use photoacoustic spectroscopy to detect pressure transients generated by NIR laser pulses, such as by using a piezoelectric device or other suitable components, or to receive laser data in other suitable manners. Laser receiver system 402 is further configured to generate digital data representing received laser data, such as PA data values that are converted to digital data and stored in one or more data memory devices.

Glucose monitor system 404 can be implemented using one or more integrated digital or analog devices in silicon, gallium arsenide, CMOS or other suitable materials, and can be configured to converts measured laser data into glucose readings, such discussed and described herein. In one example embodiment, glucose monitor system 404 can convert PA data to a glucose value, such as by using a look-up table in a read-only memory device to convert PA data to glucose data or in other suitable manners.

Data encoder system 406 encodes glucose readings, system status data and other suitable data and generates formatted data for transmission by Bluetooth transmitter system 408. In one example embodiment, data encoder system 406 can identify critical data, such as data that indicates a dangerous glucose reading, an incipient component failure or other critical data, and can prioritize that data for transmission. In another example embodiment, data encoder system 406 can store data until a predetermined data message size is reached, and can provide the formatted data to Bluetooth transmitter system 408 at that time or in other suitable manners.

Bluetooth transmitter system 408 forms data frames and transmits data in accordance with the Bluetooth data transmission standards. In one example embodiment, Bluetooth transmitter system 408 can be configured to use predetermined transmission frequencies and protocols to enable a Bluetooth transceiver to receive and process the data for use by a software application operating on an associated user device. Likewise, LORA, Zigbee, 802.11-compliant standards or other suitable low power and low data rate telemetry systems can also or alternatively be used.

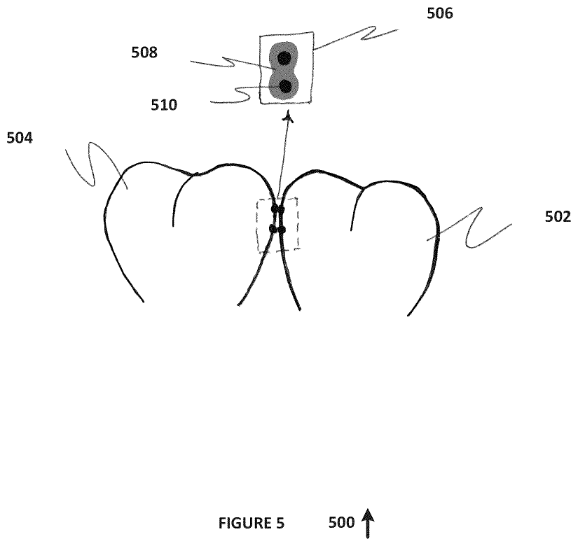

FIG. 5 is a diagram 500 of a contact area of an implant and natural tooth, which can be configured to provide power and means of direct communication, in accordance with an example embodiment of the present disclosure. Diagram 500 includes implant crown 502, porcelain crown of natural tooth 504, contact site 506, direct communication point of contact 510 and sealer 508. Direct communication point of contact 510 can be used to transfer data and energy between an implant and a live tooth, such as where the NIR laser and PA detector are installed on the live tooth and the Bluetooth transmitter, piezoelectric power devices and capacitor or battery are provided in the implant, or in other suitable embodiments.

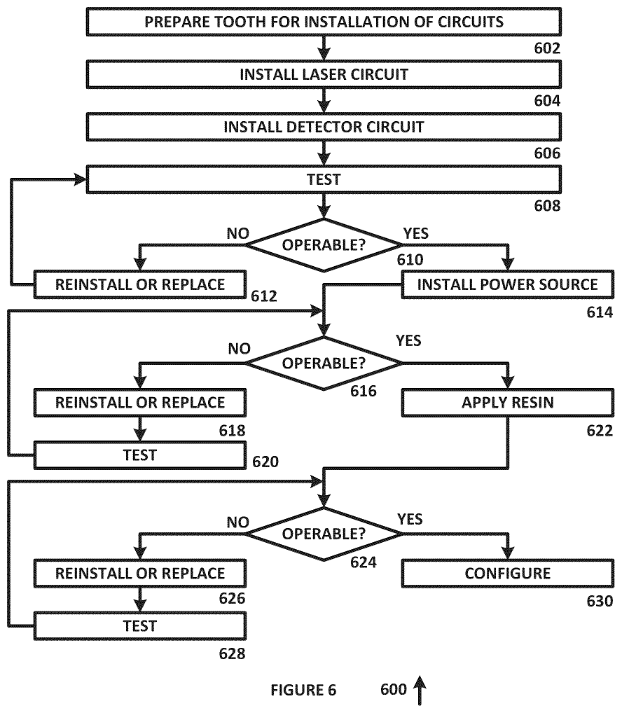

FIG. 6 is a diagram of an algorithm 600 for configuration of components for monitoring blood glucose levels in vivo, in accordance with an example embodiment of the present disclosure. Algorithm 600 can be implemented in hardware or a suitable combination of hardware and software.

Algorithm 600 begins at 602, where the tooth is prepared for installation of circuits. In one example embodiment, the tooth can be cleaned and enamel, dentin and other tooth materials can be removed in a location where circuits will be installed. The prepared tooth can then be sterilized and other suitable procedures can also or alternatively be used. The algorithm then proceeds to 604.

At 604, a laser circuit is installed on the tooth. In one example embodiment, the laser circuit can be an integrated circuit on a silicon substrate or other suitable materials, and can include one or more NIR laser devices or other suitable components that can be used to detect specific molecules that are present in the blood that enters the tooth. The laser circuit can be secured to the tooth using epoxy or other suitable materials. The algorithm then proceeds to 606.

At 606, a detector circuit is installed. In one example embodiment, the detector circuit can be an integrated circuit on a silicon substrate or other suitable materials, and can include one or more PA devices or other suitable components that can be used to detect specific molecules that are present in the blood that enters the tooth. The detector circuit can be secured to the tooth using epoxy or other suitable materials, opposite from the laser circuit or in other suitable configurations. The algorithm then proceeds to 608.

At 608, the installed components are tested for operability. In one example embodiment, a temporary power source can be connected to the laser circuit and the detector circuit, and one or more test procedures can be used to determine whether the laser circuit and detector circuit are operating properly, such as by applying power pulses to the laser circuit and determining whether the detector circuit makes corresponding measurements, or in other suitable manners. The algorithm then proceeds to 610.

At 610, it is determined whether the laser circuit and detector circuit are operable, such as by using automated test equipment or in other suitable manners. If it is determined that the laser circuit, the detector circuit, or both circuits are not operable, the algorithm proceeds to 612. If it is determined that the circuits are operable, the algorithm proceeds to 614.

At 612, the laser circuit, the detector circuit or both circuits are reinstalled or replaced. In one example embodiment the laser circuit and detector circuit can be reinstalled, where the circuits are operable but no detection of a generated pulse is made by the detector circuit. In another example embodiment, the circuits can be replaced if they are inoperable, or other suitable processes can also or alternatively be implemented. The algorithm then returns to 608.

At 614, the power source for the laser circuit and detector circuit is installed. In one example embodiment, the power source can be installed on the same tooth as the laser circuit and the detector circuit, and one or more conductors or other suitable power conducting structures or materials can be installed between the power source and the laser circuit and detector circuit. In another example embodiment, the power source can be installed on an adjacent tooth and one or more connectors can be disposed between the teeth to provide power to the laser circuit and the detector circuit, or other suitable configurations can also or alternatively be used. The algorithm then proceeds to 616.

At 616, it is determined whether the laser circuit, the detector circuit and the power source are operable. In one example embodiment, automated test equipment or circuits can be used to test operability, where an operable configuration will generate one or more predetermined responses to one or more input signals. If it is determined that the laser circuit, the detector circuit, the power source, or two or more components are not operable, the algorithm proceeds to 618, otherwise the algorithm proceeds to 622.

At 618, the laser circuit, the detector circuit, the power circuit, or two or more of the components are reinstalled or replaced. In one example embodiment, the components can be repositioned, one or more components can be replaced, or other suitable processes can also or alternatively be implemented. The algorithm then proceeds to 620.

At 620, the new configuration is tested, and the algorithm then returns to 616.

At 622, resin is applied to the components In one example embodiment, the resin can be applied over the laser circuit, the detector circuit, the power source and any power conductors, or other suitable processes can be used to protect the components from the environment The power source components can also or alternatively be protected, or implant procedures can be used where the power source is in an adjacent tooth. The algorithm then proceeds to 624.

At 624, it is determined whether the configured components are operable. In one example embodiment automated test equipment or other suitable processes can be used to evaluate operability. If it is determined that the configured components are not operable, the algorithm proceeds to 626, otherwise the algorithm proceeds to 630.

At 626, one or more of the installed components are reinstalled or replaced. In one example embodiment, the components can be repositioned, one or more components can be replaced, or other suitable processes can also or alternatively be implemented. The algorithm then proceeds to 628.

At 628, the reinstalled or replaced components are tested. The algorithm then returns to 624.

At 630, the components are configured for continuous operation. In one example embodiment, additional configuration data can be installed in one or more memory devices, and adjunct wireless device can be configured or other suitable processes can also or alternatively be used.

In operation, algorithm 600 allows components for monitoring blood glucose levels to be configured for use in vivo. While algorithm 600 is shown as a flow chart, a person of skill will recognize that it can also or alternatively be implemented as a state diagram, a ladder diagram, as one or more objects or in other suitable manners.

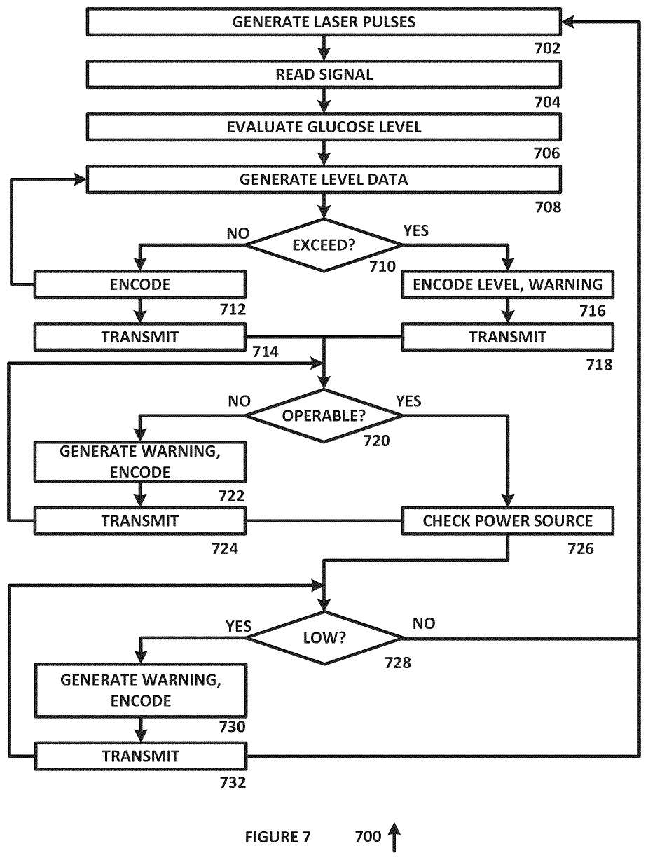

FIG. 7 is a diagram of an algorithm 700 for monitoring blood glucose levels in vivo, in accordance with an example embodiment of the present disclosure. Algorithm 700 can be implemented in hardware or a suitable combination of hardware and software.

Algorithm 700 begins at 702, where laser pulses are generated. In one example embodiment, the laser pulses are generated by an NIR laser device in a laser circuit that has been installed on a tooth when a predetermined power level has been reached at a power storage device, such as to use the stored energy before the storage capacity is reached and additional energy is wasted, or in other suitable embodiments. The algorithm then proceeds to 704.

At 704, the laser signal is read at a detector circuit. In one example embodiment, a PA detector can be used to detect signals generated by NIR laser excitation of glucose molecules. Likewise, other suitable molecules and laser excitation sources can also or alternatively be used to detect other molecules. The algorithm then proceeds to 706.

At 706, glucose levels are evaluated. In one example embodiment, the glucose levels can be determined by cross-referencing a firmware table of glucose levels that correspond to PA detector readings, or other suitable processes can also or alternatively be used. The algorithm then proceeds to 708.

At 708, level data is generated. In one example embodiment, data representing a glucose level can be stored in a data buffer for subsequent transmission, or other suitable processes can also or alternatively be used. The algorithm then proceeds to 710.

At 710, it is determined whether the level data exceeds a predetermined value. In one example embodiment, the level can be fixed, variable, can change over time, can change as a function of the derivative of the change in the readings, or other suitable level data can also or alternatively be used. If it is determined that the level data does not exceed a predetermined value, the algorithm proceeds to 712, otherwise the algorithm proceeds to 716.

At 712, the level data is encoded for transmission. In one example embodiment, data representing a glucose level can be retrieved from the data buffer and transferred to a transmission buffer, such as when sufficient energy for transmission has been stored, at a predetermined time, or other suitable processes can also or alternatively be used. The algorithm then proceeds to 714.

At 714, the data is transmitted. In one example embodiment, the data can be transmitted using a Bluetooth data transmission standard or other suitable data transmission standards. The algorithm then returns to 702.

At 716, the glucose level and any associated glucose level warnings are encoded for transmission. In one example embodiment, data representing a glucose level can be retrieved from the data buffer and transferred to a transmission buffer, such as when sufficient energy for transmission has been stored, at a predetermined time, or other suitable processes can also or alternatively be used. The algorithm then proceeds to 718.

At 718, the data is transmitted. In one example embodiment, the data can be transmitted using a Bluetooth data transmission standard or other suitable data transmission standards. The algorithm then returns to 702.

At 720, it is determined whether the laser circuit, the detector circuit, the power source or other components are operable. In one example embodiment, circuit parameters such as voltages, currents, impedance, charge decay time or other suitable parameters can be monitored and an indicator can be generated if a voltage, current, impedance, charge decay time or other suitable parameters fall outside of expected values. If it is determined that one or more components are not operable, the algorithm proceeds to 722, otherwise the algorithm proceeds to 726.

At 722, warning data is generated and encoded for transmission. In one example embodiment, when a voltage level drops below a designed value, a detector circuit such as a transistor can switch off, resulting in a state change from a logical 1 to a logical 0, or other suitable parameters can be used to cause an encoder to generate a data value that represents a corresponding operational condition. The algorithm then proceeds to 724.

At 724, the data is transmitted. In one example embodiment, the data can be transmitted using a Bluetooth data transmission standard or other suitable data transmission standards. The algorithm then proceeds to 726.

At 726, a power source is monitored for malfunctioning. In one example embodiment, a sensor can be used to monitor the voltage, current, charge time, discharge time or other parameters associated with a power source. The algorithm then proceeds to 728.

At 728, it is determined whether the power source has lost operational stability, such as if the no load voltage is low, if the stored charge is low or if other normal operating parameters are out of range. If the power supply is operational, the algorithm returns to 702, otherwise the algorithm proceeds to 730.

At 730, warning data associated with the power supply condition is generated and encoded for transmission. The algorithm then proceeds to 732.

At 732, the encoded warning data is transmitted. In one example embodiment, the data can be transmitted using a Bluetooth data transmission standard or other suitable data transmission standards. The algorithm then returns to 702.

In operation, algorithm 700 allows for monitoring blood glucose levels to be configured for use in vivo. While algorithm 700 is shown as a flow chart, a person of skill will recognize that it can also or alternatively be implemented as a state diagram, a ladder diagram, as one or more objects or in other suitable manners.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A system for monitoring blood content, comprising:
an integrated laser circuit mounted on a tooth;
an integrated detector circuit mounted on the tooth at a different location from the integrated laser circuit; and
a power supply coupled to the integrated laser circuit and the integrated detector circuit.

2. The system of claim 1 wherein the integrated laser circuit comprises a near-infrared laser.

3. The system of claim 1 wherein the integrated laser circuit comprises a tuned laser frequency to excite a predetermined molecule.

4. The system of claim 1 wherein the integrated laser circuit comprises a silicon substrate.

5. The system of claim 1 wherein the integrated detector circuit comprises a laser photoacoustic spectroscopy detector.

6. The system of claim 1 wherein the integrated detector circuit comprises a silicon substrate.

7. The system of claim 1 wherein the integrated laser circuit comprises a first tuned laser frequency to excite a first predetermined molecule and a Second tuned laser frequency to excite a second predetermined molecule.

8. The system of claim 1 wherein the power supply comprises a plurality of piezoelectric devices disposed on a substrate.

9. The system of claim 8 wherein the piezoelectric devices are disposed on a tooth surface to generate energy during mastication.

10. The system of claim 1 wherein the integrated detector circuit comprises a wireless transmitter configured to transmit wireless data.

11. The system of claim 1 wherein the integrated detector circuit comprises a glucose measurement system configured to determine a blood glucose level.

12. The system of claim 1 wherein the integrated detector circuit comprises a data encoder configured to receive and encode data.

13. The system of claim 1 wherein the integrated detector circuit comprises a data memory device configured to receive and store data.

14. The system of claim 1 wherein the integrated laser circuit comprises a data memory device configured to receive and store data.

15. The system of claim 1 wherein the integrated laser circuit comprises a laser status system configured to detect a laser failure parameter.

16. The system of claim 1 wherein the integrated laser circuit comprises a power status system configured to detect a power failure parameter.

17. A system of generating energy in vivo, comprising:
a plurality of piezoelectric devices disposed on a substrate; and
wherein the substrate is disposed on a tooth surface and is configured to generate energy during mastication.

18. The system of claim 17 further comprising a battery coupled to the piezoelectric devices.

19. The system of claim 17 further comprising a super capacitor coupled to the piezoelectric devices.

20. The system of claim 17 wherein the battery is disposed inside of an implant.
